SEQUENCE LISTING (15 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - DNA, 534 bp, Homo sapiens
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 3 - DNA, 1380 bp, Homo sapiens
atgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 4 - DNA, 1110 bp, Homo sapiens
atgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 5 - DNA, 699 bp, Homo sapiens
atggggcggctggttctgctgtggggagctgccgtctttctgctgggaggctggatggctttggggcaaggaggagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagacacagtcccgctctgtcacccaggctggagtgcagtggtgcgatctctgcttgctacaaccttcgcctcccaggttcaagcgattctcctgcctcagcctcccaagtagctgggattacaggcacccgccaccacgcctggctaatttttgtattatcagtagagacggggtttctccatgttggccaggctggtctcgaacttgcgacctcaggtga

SEQ ID NO: 6 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 7 - DNA, 480 bp, Homo sapiens
atgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 8 - protein, 324 aa, Homo sapiens
MTILGTTFGMVFSLLQVVSGESGYAQNGDLEDAELDDYSFSCYSQLEVNGSQHSLTCAFEDPDVNTTNLEFEICGALVEVKCLNFRKLQEIYFIETKKFLLIGKSNICVKVGEKSLTCKKIDLTTIVKPEAPFDLSVIYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGLSLSYGPVSPIIRRLWNIFVRNQEKIRLIBZVAGHANPRVISINALAPPSILRMSTREATMENTSEQENCESDCGDRNMBERSDCGDR

SEQ ID NO: 9 - DNA, 20 bp, artificial sequence
  synthetic
catctttgtaagaaaccaag

SEQ ID NO: 10 - DNA, 20 bp, artificial sequence
  Synthetic
tggcagtccaggaaactttc

SEQ ID NO: 11 - DNA, 21 bp, artificial sequence
  Synthetic
tggagaaagtggctatgctca

SEQ ID NO: 12 - DNA, 23 bp, artificial sequence
  Synthetic
caaccttcacacatatattgctc

SEQ ID NO: 13 - DNA, 30 bp, artificial sequence
  Synthetic
agctccaactgcccatctgaggatgtagtc

SEQ ID NO: 14 - DNA, 27 bp, artificial sequence
  Synthetic
gtgcttttgaggacccagatgtcaaca

SEQ ID NO: 15 - DNA, 24 bp, artificial sequence
  Synthetic
tccagagatcaataatagctcagg

(19) United States
(12) Patent Application Publication
(10) Pub. No.: US 2006/0198822 A1
(43) Pub. Date: Sep. 7, 2006

(54) Treatment for multiple sclerosis

(75) Inventors: David Richmond Booth, Chatswood West (AU); Graeme John Stewart, Palm Beach (AU)

Correspondence Address: KLARQUIST SPARKMAN, LLP, 121 SW SALMON STREET, SUITE 1600, PORTLAND, OR, 97204, US

(73) Assignee: Sydney West Area Health

(21) Appl. No.: 11/215,866

(22) Filed: Aug. 30, 2005

(30) Foreign Application Priority Data
Mar. 2, 2005 (AU) 2005900974

(51) Int. Cl. A61K 38/20; A61K 35/14

(52) U.S. Cl. 424/85.2; 424/93.7

(57) ABSTRACT

It is disclosed herein that particular forms of MS have significant pathogenetic differences both between each other and when compared to controls. In particular, CD127 is under-expressed in one form of MS but over-expressed in another form, relative both to each form of MS and to controls. Methods and compositions are provided for the treatment and/or diagnosis of disease caused by forms of multiple sclerosis that under-express and forms that over-express CD127. In specific examples, the methods for treating CD127-low MS comprise administering an effective amount of IL-7 or an effective amount of leukocytes treated with IL-7. Also provided are methods for treating CD127-low MS wherein leukocytes are induced to express at least one receptor, a subunit of which is CD127.

### TECHNICAL FIELD

The present invention relates to methods and compositions for the treatment and/or diagnosis of disease caused by forms of multiple sclerosis that under-express and over-express CD127.

### BACKGROUND OF THE INVENTION

Multiple sclerosis (MS) is a devastating neurodegenerative disease that affects approximately 1,100,000 people worldwide, particularly young adults (Pugliatti et al. (2002)). It is the most common demyelinating disease of the central nervous system, resulting in sclerotic plaques and axonal damage, and yet its etiology remains unknown.

One of the reasons underlying the lack of progress in thoroughly characterizing and therefore treating MS is the marked variability and unpredictability in clinical progression. Neurological signs associated with MS encompass a wide array of symptoms including limb weakness, compromised motor and cognitive function, sensory impairment, bladder disorders, sexual dysfunction, fatigue, ataxia, deafness and dementia.

Despite such variation in symptoms, the progression of several clinical courses has been classified. The majority of patients with MS follow a relapsing-remitting course in the early stages of the disease, characterised by increased severity of existing symptoms and the appearance of new symptoms, followed by variable periods of total or partial recovery. Such relapsing-remitting MS (RRMS) may be inactive for several years between distinct attacks. However, most patients with RRMS ultimately enter a secondary chronic progressive phase, characterised by progressive disability and classified as secondary progressive MS (SPMS). This disease state may also involve relapses, thereby known as relapsing progressive MS (RPMS).

While both SPMS and RPMS are pre-empted by RRMS, a further distinct classification of the disease involves a gradual worsening of symptom severity over time without initial intermittent relapses. This form of MS, known as primary chronic progressive MS, is variously referred to as either chronic progressive MS (CPMS) or primary progressive MS (PPMS). This form of the disease affects about 10% of patients.

Such diversity in MS progression is thought to be due at least in part to the wide array of risk factors that are suspected to cause the disease. These include genetic, immunologic and environmental factors such as infectious viruses and bacteria. In relation to genetic factors, it has been demonstrated that identical twins have a 30% chance of developing MS if one twin is affected, with fraternal twins and siblings and children of probands having a 1-2% chance; this compares with a prevalence of MS in the normal population of about 0.1% (Robertson et al. (1997), Dyment et al. (2004)). The genes responsible for this heritability have been sought by linkage and association studies, and through candidate gene analysis. The MHC Class II DRB1501 allele confers a 3-4 fold relative risk in most populations, and other associated genes have been identified with a much lower risk, but the full genetic basis for MS remains unexplained, despite extensive genomic screens (Compston and Sawcer (2002)).

In addition to linkage and association studies, an understanding of MS etiology has also been sought through the identification of genes that are differentially expressed in MS patients when compared with healthy individuals. In this regard, gene microarrays have been used to compare post-mortem transcription from MS plaque types (acute verses chronic) and plaque regions (active verses inactive) (Lock and Heller (2003)). Microarrays have also been used to examine peripheral blood mononucleocytes in RRMS patients verses controls, from patients both with and without interferon-β treatment (Sturzebecher et al. (2003)), and from CNS cells in stages of experimental allergic encephalomyelitis (EAE) in mice, an animal model of MS (Lock et al. (2002)). This work has produced a number of expected results, including the finding that pro-inflammatory, proliferation genes are up-regulated and anti-inflammatory, anti-apoptotic genes are down-regulated. Such studies have also indicated surprising potential novel targets for therapeutic application such as osteopontin (Chabas et al. 2001) and TRAIL (Wandinger et al. 2003)). However, many genes that have been identified as differentially regulated in MS patients compared with healthy individuals remain of unknown significance in MS development. As yet, these studies have failed to identify genetic differences in any genes that may affect MS susceptibility and/or progression.

The significant variability and unpredictability of symptoms and clinical progression in MS has therefore given rise to myriad different disease classifications. Although such clinical classification based on patient symptoms has proved useful in characterising disease progression, it has not enabled successful treatment of the disease. This failure points to the immediate and critical need for treatments that are specifically targeted to particular forms of MS.

Hence, in order to develop such targeted treatment regimes, there is clearly a need to classify the various disease states on the basis of characteristic molecular profiles rather than gross patient symptoms, which involve variability and unpredictability during clinical progression. While the molecular characterisation of MS disease states may broadly correlate with existing clinical classifications, this approach provides a much more refined and accurate insight into precise causative elements and therefore opens the way for the development of targeted treatment regimes.

The present invention is based on the unexpected and surprising finding that particular forms of MS have significant pathogenetic differences both between each other and when compared to controls. In particular, CD127 is under-expressed in one form of MS but over-expressed in another form, relative both to each form of MS and to controls.

### SUMMARY OF THE INVENTION

According to a first aspect of the present invention there is provided a method for treating CD127-low multiple sclerosis in a patient, the method comprising administering to the patient an effective amount of IL-7.

The IL-7 may be a polypeptide comprising the amino acid sequence as set forth in SEQ ID NO:1. The polypeptide may be administered by adoptive transfer of autologous leukocytes treated with IL-7.

The leukocytes may be T-cells.

The IL-7 may be administered in the form of a nucleic acid molecule encoding IL-7. The nucleic acid molecule may comprise the nucleotide sequence as set forth in SEQ ID NO:2. The nucleotide sequence may be located in a nucleic acid construct, operably linked to a promoter active in the patient to be treated. The nucleic acid construct may be a DNA construct. The DNA construct may be a plasmid.

According to a second aspect of the present invention there is provided a method for treating CD127-low multiple sclerosis in a patient, the method comprising administering to the patient an effective amount of leukocytes treated with IL-7.

Typically the leukocytes are obtained from the patient and are stimulated by contact with IL-7 in vitro.

According to a third aspect of the present invention there is provided a method for treating CD127-low multiple sclerosis in a patient, the method comprising administering to the patient an effective amount of leukocytes that have been induced to increase their cell surface expression of at least one receptor, a subunit of which is CD127.

The leukocytes may be T-cells.

The receptor may be the IL-7 receptor or the TSLP receptor.

Typically the leukocytes are obtained from the patient and are transformed with at least one nucleic acid molecule encoding one or more subunits comprising the IL-7 receptor and/or the TSLP receptor. The nucleic acid molecules may comprise the nucleotide sequences set forth in SEQ ID NO:3, SEQ ID NO:4 or SEQ ID NO:5.

According to a fourth aspect of the present invention there is provided a method for treating CD127-low multiple sclerosis in a patient the method comprising administering to the patient an effective amount of a nucleic acid molecule encoding at least CD127.

The nucleic acid molecule may comprise the nucleotide sequence as set forth in SEQ ID NO:3.

The method of the fourth aspect may further comprise administration to the patient of an effective amount of a nucleic acid molecule encoding the common γ-chain CD132. The nucleic acid molecule encoding CD132 may comprise the nucleotide sequence as set forth in SEQ ID NO:4. Alternatively, or in addition, the method of the fourth aspect may further comprise administration to the patient of a nucleic acid molecule encoding the thymic stromal lymphopoietin receptor (TSLP-R) chain. The nucleic acid molecule encoding the TSLP-R chain may comprise the nucleotide sequence as set forth in SEQ ID NO:5.

The nucleotide sequence(s) may be located in one or more nucleic acid constructs, operably linked to a promoter(s) active in a subject to be treated.

According to a fifth aspect of the present invention there is provided a method for treating CD127-low multiple sclerosis in a patient, the method comprising administering to the patient an effective amount of TSLP.

The TSLP may be a polypeptide comprising the amino acid sequence as set forth in SEQ ID NO:6. The polypeptide may be administered by adoptive transfer of autologous leukocytes treated with TSLP.

The TSLP may be administered in the form of a nucleic acid molecule encoding TSLP. The nucleic acid molecule may comprise the nucleotide sequence as set forth in SEQ ID NO:7. The nucleotide sequence may be located in a nucleic acid construct, operably linked to a promoter active in the patient to be treated. The nucleic acid construct may be a DNA construct. The DNA construct may be a plasmid.

According to a sixth aspect of the present invention there is provided a method for treating CD127-low multiple sclerosis in a patient, the method comprising administering to the patient an effective amount of leukocytes treated with TSLP.

Typically the leukocytes are obtained from the patient and are stimulated by contact with TSLP in vitro.

According to a seventh aspect of the present invention there is provided a method for treating CD127-high multiple sclerosis in a patient the method comprising administering to the patient an effective amount of a non-functional form or homologue of IL-7 or TSLP, wherein the non-functional form or homologue retains receptor binding ability but lacks signal transduction initiation capability.

The non-functional form of IL-7 or TSLP may be a variant, fragment or analogue of IL-7 or TSLP.

According to an eighth aspect of the present invention there is provided a method for treating CD127-high multiple sclerosis in a patient the method comprising administering to the patient an effective amount of at least one inhibitor of IL-7.

The inhibitor may be a nucleic acid-based inhibitor, a peptide-based inhibitor or a small molecule inhibitor of IL-7 or nucleic acid molecule encoding the same. The nucleic acid-based inhibitor may be a siRNA molecule or an antisense construct.

According to a ninth aspect of the present invention there is provided a method for treating CD127-high multiple sclerosis in a patient the method comprising administering to the patient an effective amount of a soluble form of the IL-7 receptor.

The soluble IL-7 receptor may be administered as one or more polypeptide subunits or nucleic acid molecules encoding the same. The CD127 polypeptide may be a soluble isoform of CD127 and comprise the amino acid sequence as set forth in SEQ ID NO:8.

According to a tenth aspect of the present invention there is provided a method for treating CD127-high multiple sclerosis in a patient the method comprising administering to the patient an effective amount of at least one inhibitor of one or more of the following: CD127, CD132, the TSLP-R chain, the IL-7 receptor and the TSLP receptor.

The inhibitor may be a nucleic acid-based inhibitor, a peptide-based inhibitor or a small molecule inhibitor. The nucleic acid-based inhibitor may be a siRNA molecule or an antisense construct.

According to an eleventh aspect of the present invention there is provided a method for treating CD127-high multiple sclerosis in a patient the method comprising administering to the patient an effective amount of at least one inhibitor of TSLP.

The inhibitor may be a nucleic acid-based inhibitor, a peptide-based inhibitor or a small molecule inhibitor. The nucleic acid-based inhibitor may be a siRNA molecule or an antisense construct.

According to a twelfth aspect of the present invention there is provided a composition for treating CD127-low multiple sclerosis, the composition comprising IL-7 together with at least one pharmaceutically acceptable carrier, diluent and/or adjuvant.

According to a thirteenth aspect of the present invention there is provided a composition for treating CD127-low multiple sclerosis, the composition comprising endogenous leukocytes together with at least one pharmaceutically acceptable carrier, diluent and/or adjuvant.

The leukocytes may be T-cells.

The leukocytes may be treated in vitro with one or more of IL-7 and TSLP. The leukocytes may be treated in vitro to increase their cell surface expression of at least one receptor, a subunit of which is CD127.

According to a fourteenth aspect of the present invention there is provided a composition for treating CD127-low multiple sclerosis, the composition comprising a nucleic acid molecule encoding at least CD127 together with at least one pharmaceutically acceptable carrier, diluent and/or adjuvant.

According to a fifteenth aspect of the present invention there is provided a composition for treating CD127-low multiple sclerosis, the composition comprising TSLP together with at least one pharmaceutically acceptable carrier, diluent and/or adjuvant.

According to a sixteenth aspect of the present invention there is provided a composition for treating CD127-high multiple sclerosis, the composition comprising a non-functional form of IL-7 or TSLP, or a non-functional homologue of IL-7 or TSLP, together with at least one pharmaceutically acceptable carrier, diluent and/or adjuvant.

According to a seventeenth aspect of the present invention there is provided a composition for treating CD127-high multiple sclerosis, the composition comprising at least one inhibitor of IL-7 together with at least one pharmaceutically acceptable carrier, diluent and/or adjuvant.

According to an eighteenth aspect of the present invention there is provided a composition for treating CD127-high multiple sclerosis, the composition comprising a soluble isoform of CD127 together with at least one pharmaceutically acceptable carrier, diluent and/or adjuvant.

According to a nineteenth aspect of the present invention there is provided a composition for treating CD127-high multiple sclerosis, the composition comprising at least one inhibitor of one or more of the following: CD127, CD132, the TSLP-R chain, the IL-7 receptor and the TSLP receptor, together with at least one pharmaceutically acceptable carrier, diluent and/or adjuvant.

According to a twentieth aspect of the present invention there is provided a composition for treating CD127-high multiple sclerosis, the composition comprising at least one inhibitor of TSLP together with at least one pharmaceutically acceptable carrier, diluent and/or adjuvant.

According to a twenty-first aspect of the present invention there is provided a method for diagnosing or characterising a multiple sclerosis subtype in an individual, the method comprising the steps of:

(a) obtaining a biological sample from the individual; and

(b) assaying for the expression of CD127 in the sample.

According to a twenty-second aspect of the present invention there is provided a method for determining the susceptibility of an individual to multiple sclerosis, the method comprising the steps of:

(a) obtaining a biological sample from the individual; and

(b) assaying for the expression of CD127 in the sample.

### Definitions

In the context of this specification, the term “comprising” means “including principally, but not necessarily solely”. Furthermore, variations of the word “comprising”, such as “comprise” and “comprises”, have correspondingly varied meanings.

As used herein the terms “treating” and “treatment” refer to any and all uses which remedy a condition or symptoms, prevent the establishment of a condition or disease, or otherwise prevent, hinder, retard, or reverse the progression of a condition or disease or other undesirable symptoms in any way whatsoever.

As used herein the term “effective amount” includes within its meaning a non-toxic but sufficient amount of an agent or compound to provide the desired effect. The exact amount required will vary from subject to subject depending on factors such as the species being treated, the age and general condition of the subject, the particular agent being administered and the mode of administration and so forth. Thus, it is not possible to specify an exact “effective amount”. However, for any given case, an appropriate “effective amount” may be determined by one of ordinary skill in the art using only routine experimentation.

In the context of this specification, the term “inhibitor” refers to any agent or action capable of inhibiting expression or activity. Accordingly the inhibitor may operate directly or indirectly, or alternatively act via the direct or indirect inhibition of any one or more components of a signal transduction pathway. Such components may be molecules activated, inhibited or otherwise modulated prior to, in conjunction with, or as a consequence of protein activity. Thus, the inhibitor may operate to prevent transcription, translation, post-transcriptional or post-translational processing or otherwise inhibit the activity of a protein or a component of a signal transduction pathway in any way, via either direct or indirect action. The inhibitor may for example be nucleic acid, peptide, any other suitable chemical compound or molecule or any combination of these. Additionally, it will be understood that in indirectly impairing the activity of a protein or a component of an associated signal transduction pathway, the inhibitor may affect the activity of other cellular molecules which may in turn act as regulators of the molecule itself. Similarly, the inhibitor may affect the activity of molecules which are themselves subject to regulation or modulation by a protein or a component of an associated signal transduction pathway.

As used herein the term “polypeptide” means a polymer made up of amino acids linked together by peptide bonds. The terms “polypeptide” and “protein” are used interchangeably herein, although for the purposes of the present invention a “polypeptide” may constitute a portion of a full length protein.

As used herein, the term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. The term “nucleic acid molecule” is used interchangeably with the term “polynucleotide”.

As used herein the term “MS” refers to any form of multiple sclerosis or other disease involving one or more symptoms characteristically associated with multiple sclerosis.

As used herein the term “CD127” refers to the molecule CD127, otherwise known as IL-7R α-chain, or its precursors or derivatives thereof. Also encompassed within the scope of the invention are homologues or mimetics of CD127 which possess qualitative biological activity in common with the full-length mature activated CD127. Further, the present invention contemplates not only use of the CD127 polypeptide, but also polynucleotides encoding the same.

As used herein the term “CD132” refers to the molecule CD132, otherwise known as the common γ-chain, or its precursors or derivatives thereof. Also encompassed within the scope of the invention are homologues or mimetics of CD132 which possess qualitative biological activity in common with the full-length mature activated CD132. Further, the present invention contemplates not only use of the CD132 polypeptide, but also polynucleotides encoding the same.

As used herein the term “IL-7” refers to interleukin-7 or its precursors or derivatives thereof. Also encompassed within the scope of the invention are homologues or mimetics of IL-7 which possess qualitative biological activity in common with the full-length mature activated IL-7.

As used herein the terms “IL-7R” and “IL-7 receptor” refer to the IL-7 receptor multimeric protein complex, comprising CD127 (otherwise known as the IL-7R α-chain) and CD132 (otherwise known as the common γ-chain), or its precursors or derivatives thereof. Also encompassed within the scope of the invention are homologues or mimetics of IL-7R which possess qualitative biological activity in common with IL-7R.

As used herein the term “soluble” as it pertains to the IL-7 receptor means any form of the receptor that retains the ability to bind a ligand but is not membrane-bound and is therefore unable to initiate signal transduction as a result of ligand binding.

As used herein the term “TSLP” refers to thymic stromal lymphopoietin or its precursors or derivatives thereof. Also encompassed within the scope of the invention are homologues or mimetics of TSLP which possess qualitative biological activity in common with the full-length mature activated TSLP.

As used herein the terms “TSLPR” and “TSLP receptor” refer to the TSLP receptor multimeric protein complex, comprising CD127 (otherwise known as the IL-7R α-chain) and the TSLP-R chain, or its precursors or derivatives thereof. Also encompassed within the scope of the invention are homologues or mimetics of TSLPR which possess qualitative biological activity in common with TSLPR.

As used herein the term “CD127-low” refers to a disorder or condition associated with, at least in part, under-expression of CD127, where such under-expression is relative as compared to a basal level of expression of CD127 within the general population, or in a control sample of non-MS sufferers.

As used herein the term “CD127-high” refers to a disorder or condition other than RRMS associated with, at least in part, over-expression of CD127 where such over-expression is relative as compared to a basal level of expression of CD127 within the general population, or in a control sample of non-MS sufferers.

### BRIEF DESCRIPTION OF THE DRAWINGS AND SEQUENCE LISTING

Embodiments of the present invention will now be described, by way of example only, with reference to the accompanying drawings.

FIG. 1 (a): Comparison of genes differentially expressed: The left circle represents the comparison of the combined PPMS and SPMS groups (PPMS+SPMS) and reference groups (Con). 98 genes were under-expressed and 63 genes were over-expressed. The right circle in each pair is the comparison of the control to the reference group. The overlap represents genes shared between the controls and PPMS+SPMS compared to the reference group. (b): Comparison of MS subgroups: few genes were shared between the over- and under-expressed genes between SPMS and PPMS.

FIG. 2: Fold change in CD127 gene expression in microarray and quantitative RT-PCR: Fold changes were significantly different (Students t-test) between PPMS and SPMS samples in both microarray and quantitative RT-PCR analysis. Standard error bars are shown.

FIG. 3: Comparison of relative CD127 haplotype expression ex vivo using the cDNA primer extension assay: for (a): haplotypes tagged by coding SNPs aa46 and aa336, and (b): for all haplotypes. Expression was compared by Mann Whitney U-tests. C: control samples; MS: MS samples; ns: not significant.

FIG. 4: Expression of soluble and insoluble isoforms of CD127 mRNA: Expression levels were compared using unpaired t-tests. Individuals were homozygous for −504 T or C, corresponding to haplotype GCA (if C) or the three other haplotypes if T.

FIG. 5: Effect of CD127 genotype on CD127 expression: CD127 expression levels were compared between different −504 CD127 genotypes in CD4⁺ cells of PPMS patients. The CD127 −504 CT and TT genotypes prevalent in PPMS had lower CD127 expression than CD127 −504 CC genotypes.

FIG. 6: CD127 expression is reduced in Treg cells: flow cytometry analysis of CD127 expression levels between T cells generally and Tregs demonstrated that CD127 expression is reduced in Treg cells.

FIG. 7: CD127 expression is reduced in NKT cells: flow cytometry analysis of CD127 expression levels between T cells generally and NKTs demonstrated that CD127 expression is reduced in NKT cells.

FIG. 8: Analysis of number of CD4⁺ CD25^{hi} cells in PPMS: CD4⁺ CD25^{hi} cells (Tregs) from peripheral blood of PPMS patients were analysed by flow cytometry and examined for levels of CD127 expression. There was no difference in Treg cell numbers between PPMS patients and healthy controls.

FIG. 9: Analysis of number of CD3⁺ CD56⁺ cells in PPMS: CD3⁺ CD56⁺ cells (NKTs) from peripheral blood of PPMS patients were analysed by flow cytometry and examined for levels of CD127 expression, showing that CD3⁺ CD56⁺ cell numbers were lower in PPMS patients than in healthy controls.

FIG. 10: Increasing IL7 concentration increases cell proliferation at set concentrations of IL2: CD4⁺ CD25^{hi} cells (Tregs) from peripheral blood of a healthy control were examined for CD127 expression based on stimulation with IL2, IL7 and anti-CD3/anti-CD28 microbeads.

FIG. 11: IL7 induces proliferation of T cell subsets in vitro: CD4⁺ CD25^{hi} cells (Tregs) from peripheral blood of a PPMS patient were examined for CD127 expression based on stimulation with IL2, IL7 and anti-CD3/anti-CD28 microbeads.

The amino acid sequence set forth in SEQ ID NO:1 is the amino acid sequence of human IL-7.

The nucleotide sequence set forth in SEQ ID NO:2 is the nucleotide sequence of the gene encoding human IL-7.

The nucleotide sequence set forth in SEQ ID NO:3 is the nucleotide sequence of the gene encoding human CD127.

The nucleotide sequence set forth in SEQ ID NO:4 is the nucleotide sequence of the gene encoding human CD132.

The nucleotide sequence set forth in SEQ ID NO:5 is the nucleotide sequence of the gene encoding human TSLP receptor chain.

The amino acid sequence set forth in SEQ ID NO:6 is the amino acid sequence of human TSLP.

The nucleotide sequence set forth in SEQ ID NO:7 is the nucleotide sequence of the gene encoding human TSLP.

The amino acid sequence set forth in SEQ ID NO:8 is the amino acid sequence of the soluble isoform of human CD127.

### Modes of Performing the Invention

The IL-7 receptor is a multimeric protein complex that is expressed on the surface of T-cells from the early stages of immune repertoire development. The subunits of the IL-7 receptor comprise CD127 and CD132. CD127 is otherwise known as the IL-7 receptor α-chain and CD132 is otherwise known as the common y-chain. The IL-7 receptor usually exists as a membrane-bound molecule, tethered to the cell surface by a trans-membrane domain emanating from the CD127 protein subunit. However, a soluble, secreted form of the IL-7 receptor can be produced through cleavage and processing of the transmembrane domain. The ligand for the IL-7 receptor is the cytokine IL-7, which, in combination with other members of the cytokine family, functions as a haematopoietic growth factor to cause activation and proliferation of early lymphoid T-cells.

In addition to its role as a subunit of the IL-7 receptor complex, the CD127 protein is also a subunit of the TSLP receptor complex. This heterodimeric complex, comprising both CD127 and the thymic stromal lymphopoietin receptor chain (TSLP-R), is expressed primarily on monocytes and myeloid-derived dendritic cells and is thought to play a role in allergy and inflammation. The ligand for the TSLP receptor is TSLP, a haematopoietic protein that is expressed in the heart, liver and prostate, and which overlaps in its biological activities with IL-7. TSLP, similarly to IL-7, induces phosphorylation of STAT3 and STAT5 upon binding to its receptor, but uses kinases other than the JAKs for activation.

The inventors have made the surprising and unexpected discovery that CD127 is under-expressed in some forms of multiple sclerosis (MS) and over-expressed in other forms of MS, relative to controls. This finding was made as a result of investigations by the inventors into potential treatments for MS that would be specifically targeted to particular forms of the disease.

In the course of their investigation, the present inventors developed an original experimental protocol that departed from conventional drug development methodology. In part, this original protocol incorporated the notion that aberrant gene expression profiles in patients with MS may be an effect of the disease rather than a cause of the disease. In this case, examination of gene transcription profiles would not necessarily indicate therapeutic targets, as any dysregulated gene profiles may indicate either a cause or an effect of the disease state. Hence, the protocol developed by the inventors incorporated examining genetic differences in gene promoter regions. Such differences would therefore indicate that inherited factors were causing the gene dysregulation, thus supporting a role for any such genes in causing disease susceptibility and/or progression, as opposed to merely being a result of disease susceptibility and/or progression.

In addition, to reduce problems associated with heterogeneity due to variation in the level of disease activity, the inventors focused on the primary and secondary chronic progressive subgroups of MS patients (PPMS and SPMS), who have continuous disease, rather than the relapsing-remitting group of patients (RRMS), who are often in remission.

The results of these studies have demonstrated that patients traditionally classified as suffering from PPMS under-express CD127, and patients traditionally classified as suffering from SPMS over-express CD127, relative both to each other form of MS and to controls. This surprising result initially involved examining both biochemical pathways over-represented in dysregulated PPMS and SPMS genes, and dysregulated genes in PPMS and SPMS compared both to each other and to reference samples. After determining the identity of differentially regulated genes, the inventors investigated the population association of allelic polymorphisms in the promoter regions of those genes and elucidated common promoter polymorphisms and haplotypes in putative promoter regions of CD127. A CD127 population association study, involving an analysis of CD127 allele transmission in trio families and the frequency of CD127 alleles and genotypes in subtypes of MS, highlighted the confounding effect of heterogeneity between MS subtypes in other previous association studies in terms of analysing transmission of alleles of differentially regulated genes. Furthermore, CD127 expression profiles from different haplotypes were examined ex vivo. This led to the determination that the CD127 genotypes prevalent in PPMS also had lower CD127 expression in CD4⁺ T cells. Further analyses of regulatory T cells (Tregs) and natural killer T cells (NKTs) showed that both of these T cell subsets expressed lower levels of CD127 than T cells generally, and that impaired cell number (NKTs) and impaired cell function (Tregs) may be involved in PPMS pathogenesis. Moreover, the ligand (IL7) for the receptor of which CD127 comprises a subunit, causes Treg proliferation and synergistically augments IL2-mediated Treg proliferation.

These studies, variously involving microarray analysis, genotyping and CD127 expression profiling, also dramatically demonstrated the advantage in developing a more concise form of classifying different subtypes of MS, based not on the manifestation of gross patient symptoms characteristically associated with clinical progression, but rather on the basis of characteristic molecular profiles. While such molecular characterisation may sometimes broadly overlap with traditional clinical classifications, this approach clearly provides a significantly more refined and accurate insight into causative elements, thus paving the way for the development of treatment regimes that are specifically targeted to particular molecular subtypes of MS.

Accordingly, the present invention provides methods and compositions for the treatment of forms of MS herein classified as “CD127-low” and “CD127-high”. The different expression profiles associated with these two forms of MS are unexpected, and indicate fundamentally different treatment regimes. Indeed, in the case of CD127-low MS, these treatments are contrary to the expectation of a person skilled in the art on the basis of established dogma in the art.

Those skilled in the art will appreciate that for each of CD27-low MS and CD127-high MS the compositions and methods of treatment disclosed herein may be used in isolation or in combination. The skilled addressee will understand “combination” to mean that the methods or compositions disclosed herein may be used in conjunction with one another, or as part of a combination therapy together with alternative methods or compositions for the treatment of MS.

In one embodiment the invention provides a method for treating CD127-low MS sufferers with IL-7, thus maximizing the level of binding of IL-7 to IL-7R and compensating for under-expression of IL-7R on the T-cell surface.

In another embodiment the invention provides a method for treating CD127-low MS sufferers with one or more polynucleotides encoding a receptor, a subunit of which is CD127, thus maximizing the level of binding of the appropriate ligand to the CD127-containing receptor. The receptor may be the IL-7 receptor, composed of CD127 and CD132, or the TSLP receptor, composed of CD127 and the TSLP-R chain.

Another embodiment of the invention provides a method for treating CD127-low MS sufferers with TSLP, thus maximizing the level of binding of TSLP to the TSLP receptor.

The invention also provides a method for treating CD127-high MS sufferers with a non-functional form or non-functional homologue of IL-7 or TSLP, wherein the non-functional form or homologue retains receptor binding ability but lacks signal transduction initiation capability, thus minimizing the level of binding of functional IL-7 to IL-7R or TSLP to the TSLP receptor.

Further embodiments of the invention provide methods for treating CD127-high MS sufferers with at least one inhibitor of IL-7, thus minimizing the level of binding of IL-7 to IL-7R or TSLP to the TSLP receptor.

The invention also provides a method for treating CD127-high MS sufferers with a soluble non-functional form of IL-7R, thus minimizing the level of binding of IL-7 to membrane-bound functional IL-7R.

The invention further provides a method for treating CD127-high MS sufferers with at least one inhibitor of CD127, and optionally at least inhibitor of CD132 or TSLP-R, thus minimizing the level of binding of IL-7 to IL-7R and/or TSLP to the TSLP receptor.

The invention also provides compositions for treating CD127-low MS, comprising either IL-7, endogenous T cells, CD127 or TSLP.

The invention also provides compositions for treating CD127-high MS, comprising either a non-functional form of IL-7, an inhibitor of IL-7, a soluble isoform of CD127, an inhibitor of CD127, CD132 or IL-7R or an inhibitor of TSLP.

Embodiments of methods of the invention involve the transfer of leukocytes, typically T-cells, to a patient diagnosed with either CD127-low MS or CD127-high MS, wherein the T-cells have been appropriately treated in vitro. Typically the leukocytes have been obtained from the patient. For example, in the case of a patient suffering from CD127-low MS, T-cells may be isolated from the patient and treated with an IL-7 or TSLP polypeptide or polynucleotide encoding the same or with at least one nucleic acid molecule encoding one or more subunits of a CD127-containing receptor to increase the cell surface expression of the receptor. The autologous T-cells may then be re-introduced into the patient. In the case of a patient suffering from CD127-high MS, isolated leukocytes may be treated, for example, with one or more inhibitors of IL-7, TSLP and/or IL-7R or TSLPR or one or more subunits thereof. Methods for autologous cell transfer including the isolation, in vitro treatment and re-introduction of cells are known to those skilled in the art (see, for example, Homann, D and von Herrath, M. (2004) Regulatory T cells and type 1 diabetes. Clin Immunol 112(3); 202-9, the disclosures of which are incorporated herein by reference).

The present invention also contemplates the treatment of CD127-low CD127-high MS by gene therapy approaches. Accordingly, embodiments of the present invention provide for the administration of polynucleotides directly to an individual via gene therapy. Alternatively, T-cells isolated from the individual may be transformed with one or more polynucleotides so as to achieve the desired effect, as described above, and the T-cells subsequently re-introduced into the patient.

In particular embodiments of the invention, polynucleotides may be used as naked DNA or within in a vector. The vector may be a plasmid vector, a viral vector, or any other suitable vehicle adapted for the insertion and foreign sequences and introduction into eukaryotic cells. Typically the vector is an expression vector capable of directing the transcription of the DNA sequence of the polynucleotide into mRNA. The vector may include expression control and processing sequences such as a promoter, an enhancer, ribosome binding sites, polyadenylation signals and transcription termination sequences. Examples of suitable viral expression vectors include for example Epstein-barr virus-, bovine papilloma virus-, adenovirus- and adeno-associated virus-based vectors. The vector may be episomal.

Polypeptides and Polynucleotides

As described above the methods and compositions of the embodiments of the invention typically involve the use of one or more polypeptides or polynucleotides for receptors of T cells and their ligands. In particular, such receptors and ligands may comprise IL-7, CD127, CD132, IL-7 receptor, TSLP receptor chain and TSLP. Typically the polypeptides and polynucleotides to which the methods and compositions of the present invention relate are the human protein and gene. The amino acid sequence of the human IL-7 protein is shown in SEQ ID NO:1 (GenBank Accession No. NM_{—}000880), and the nucleotide sequence of the human IL-7 gene is shown in SEQ ID NO:2 (GenBank Accession No. NM_{—}000880). The nucleotide sequence of the human CD127 gene is shown in SEQ ID NO:3 (GenBank Accession No. NM_{—}002185). The nucleotide sequence of the human CD132 gene is shown in SEQ ID NO:4 (GenBank Accession No. NM_{—}000206). The nucleotide sequence of the human TSLP-R receptor chain gene is shown in SEQ ID NO:5 (GenBank Accession No. AK026800). The amino acid sequence of the human TSLP protein is shown in SEQ ID NO:6 (GenBank Accession No. AY037115), and the nucleotide sequence of the human TSLP gene is shown in SEQ ID NO:7 (GenBank Accession No. AY037115). The amino acid sequence of the soluble isoform of human CD127 is in SEQ ID NO: 8 (Swiss Prot: P16871-3).

According to embodiments of the invention, the disclosed polypeptides may have the amino acid sequences as set forth in the sequence listing or display sufficient sequence identity thereto to hybridise to the sequences as set forth in the sequence listing. In alternative embodiments, the nucleotide sequence of the polynucleotide may share at least 50%, 60%, 70%, 80%, 85%, 90%, 95%, 96%, 97%, 98% or 99% identity with the sequences as set forth in the sequence listing.

According to embodiments of the invention, the disclosed polynucleotides may have the nucleotide sequences as set forth in the sequence listing or display sufficient sequence identity thereto to hybridise to the nucleotide sequences as set forth in the sequence listing. In alternative embodiments, the nucleotide sequence of the polynucleotide may share at least 30%, 40%, 50%, 60%, 70%, 80%, 85%, 90%, 95%, 96%, 97%, 98% or 99% identity with the nucleotide sequences as set forth in the sequence listing.

Within the scope of the terms “protein”, “polypeptide” and “polynucleotide” as used herein are fragments and variants thereof, including but not limited to reverse compliment and antisense forms.

The term “fragment” refers to a nucleic acid or polypeptide sequence that encodes a constituent or is a constituent of a full-length protein or gene. In terms of the polypeptide the fragment possesses qualitative biological activity in common with the full-length protein.

The term “variant” as used herein refers to substantially similar sequences. Generally, nucleic acid sequence variants encode polypeptides which possess qualitative biological activity in common. Generally, polypeptide sequence variants also possess qualitative biological activity in common. Further, these polypeptide sequence variants may share at least 50%, 55%, 60%, 65%, 70%, 75%, 80%, 85%, 90%, 95%, 96%, 97%, 98% or 99% sequence identity.

Further, a variant polypeptide may include analogues, wherein the term “analogue” means a polypeptide which is a derivative of the disclosed polypeptides, which derivative comprises addition, deletion or substitution of one or more amino acids, such that the polypeptide retains substantially the same function as the native polypeptide from which it is derived. The term “conservative amino acid substitution” refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

In accordance with the present invention, fusion proteins may also be engineered to improve characteristics of a polypeptide or a variant or fragment thereof. For example, peptide moieties may be added to the polypeptide to increase stability of the polypeptide. The addition of peptide moieties of polypeptides are routine techniques well known to those of skill in the art.

An individual suffering from MS can readily be classified as suffering from CD127-low MS or CD127-high MS by determining the level of expression of CD127. CD127 expression may be determined by measuring protein expression or mRNA expression levels. As a result, an appropriate treatment regime may be recommended and implemented. Alternatively, measurement of CD127 expression levels may be used to characterise or diagnose MS in an individual, wherein a reduced level of CD127 expression relative to a control is indicative of CD127-low MS and an increased level of CD127 expression relative to a control is indicative of CD127-high MS.

Expression of polynucleotides, proteins or polypeptides may be determined by any one of a number of techniques well known to those skilled in the art. For example, expression may be determined by assaying mRNA transcript abundance in a sample. mRNA abundance may be measured, for example, by either reverse transcriptase-PCR (RT-PCR) followed by phospho-imaging analysis, or real-time RT-PCR. Alternatively expression of a protein or polypeptide may be determined using an antibody that binds to the protein or polypeptide or a fragment thereof, using a technique such as enzyme-linked immunosorbent assay (ELISA), flow cytometry or fluorescence activated cell sorting (FACS).

Inhibitors

Embodiments of the present invention provide methods and compositions for inhibiting the expression of the disclosed polypeptides and/or polynucleotides using an inhibitor thereof. Typically the inhibitor may be nucleic-acid based, peptide-based or other suitable chemical compound.

The inhibitor may be a nucleic-acid based inhibitor of expression of a polynucleotide disclosed herein or a fragment thereof. Suitable molecules include small interfering RNA (siRNA) species, antisense constructs, such as antisense oligonucleotides, and catalytic antisense nucleic acid constructs. Suitable molecules can be manufactured by chemical synthesis, recombinant DNA procedures or, in the case of antisense RNA, by transcription in vitro or in vivo when linked to a promoter, by methods known to those skilled in the art.

One suitable technology for inhibiting gene expression, known as RNA interference (RNAi), (see, eg. Chuang et al. (2000) PNAS USA 97: 4985) may be used for the purposes of the present invention, according to known methods in the art (for example Fire et al. (1998) Nature 391: 806-811; Hammond, et al. (2001) Nature Rev, Genet. 2: 110-1119; Hammond et al. (2000) Nature 404: 293-296; Bernstein et al. (2001) Nature 409: 363-366; Elbashir et al (2001) Nature 411: 494-498; WO 99/49029 and WO 01/70949, the disclosures of which are incorporated herein by reference), to inhibit the expression of the disclosed polynucleotides. RNAi refers to a means of selective post-transcriptional gene silencing by destruction of specific mRNA by small interfering RNA molecules (siRNA). The siRNA is typically generated by cleavage of double stranded RNA, where one strand is identical to the message to be inactivated. Double-stranded RNA molecules may be synthesised in which one strand is identical to a specific region of the mRNA transcript and introduced directly. Alternatively corresponding dsDNA can be employed, which, once presented intracellularly is converted into dsRNA. Methods for the synthesis of suitable siRNA molecules for use in RNAi and for achieving post-transcriptional gene silencing are known to those of skill in the art. The skilled addressee will appreciate that a range of suitable siRNA constructs capable of inhibiting the expression of the disclosed polynucleotides can be identified and generated based on knowledge of the sequence of the gene in question using routine procedures known to those skilled in the art without undue experimentation.

Those skilled in the art will appreciate that there need not necessarily be 100% nucleotide sequence match between the target sequence and the siRNA sequence. The capacity for mismatch is dependent largely on the location of the mismatch within the sequences. In some instances, mismatches of 2 or 3 nucleotide may be acceptable but in other instances a single nucleotide mismatch is enough to negate the effectiveness of the siRNA. The suitability of a particular siRNA molecule may be determined using routine procedures known to those skilled in the art without undue experimentation.

Sequences of antisense constructs may be derived from various regions of the target gene. Antisense constructs may be designed to target and bind to regulatory regions of the nucleotide sequence, such as the promoter, or to coding (exon) or non-coding (intron) sequences. Antisense constructs of the invention may be generated which are at least substantially complementary across their length to a region of the gene in question. Binding of an antisense construct to its complementary cellular sequence may interfere with transcription, RNA processing, transport, translation and/or mRNA stability.

Antisense constructs of the present invention may be derived from the human CD127 gene, or non-human animal variants thereof. For example, antisense constructs derived from non-human genes having at least 50% sequence identity with the human gene can be used in the methods of the invention. Non-human CD127 genes may have at least 60%, at least 70%, at least 80% or at least 90% sequence identity with their human homologue.

Suitable antisense oligonucleotides may be prepared by methods well known to those of skill in the art. Typically antisense oligonucleotides will be synthesized on automated synthesizers. Suitable antisense oligonucleotides may include modifications designed to improve their delivery into cells, their stability once inside a cell, and/or their binding to the appropriate target. For example, the antisense oligonucleotide may be modified by the addition of one or more phosphorothioate linkages, or the inclusion of one or morpholine rings into the backbone.

In particular embodiments of the invention, suitable inhibitory nucleic acid molecules may be administered in a vector. The vector may be a plasmid vector, a viral vector, or any other suitable vehicle adapted for the insertion of foreign sequences and introduction into eukaryotic cells. Preferably the vector is an expression vector capable of directing the transcription of the DNA sequence of an inhibitory nucleic acid molecule of the invention into RNA. Viral expression vectors include, for example, epstein-barr virus-, bovine papilloma virus-, adenovirus- and adeno-associated virus-based vectors. In one embodiment, the vector is episomal. The use of a suitable episomal vector provides a means of maintaining the inhibitory nucleic acid molecule in target cells in high copy number extra-chromosomally thereby eliminating potential effects of chromosomal integration.

A further means of substantially inhibiting gene expression may be achieved by introducing catalytic antisense nucleic acid constructs, such as ribozymes, which are capable of cleaving RNA transcripts and thereby preventing the production of wildtype protein. Ribozymes are targeted to and anneal with a particular sequence by virtue of two regions of sequence complementarity to the target flanking the ribozyme catalytic site. After binding, the ribozyme cleaves the target in a site-specific manner. The design and testing of ribozymes which specifically recognize and cleave sequences of interest can be achieved by techniques well known to those in the art (for example Lieber and Strauss, (1995) Mol. Cell. Biol. 15:540-551, the disclosure of which is incorporated herein by reference).

Alternative inhibitors of polypeptides disclosed herein may include antibodies. Suitable antibodies include, but are not limited to, polyclonal antibodies, monoclonal antibodies, chimeric antibodies, humanised antibodies, single chain antibodies and Fab fragments.

Antibodies may be prepared from discrete regions or fragments of the polypeptide of interest. An antigenic polypeptide contains at least about 5, and preferably at least about 10, amino acids. Methods for the generation of suitable antibodies will be readily appreciated by those skilled in the art. For example, a suitable monoclonal antibody, typically containing Fab portions, may be prepared using the hybridoma technology described in Antibodies—A Laboratory Manual, Harlow and Lane, eds. Cold Spring Harbor Laboratory, N.Y. (1988), the disclosure of which is incorporated herein by reference.

Similarly, there are various procedures known in the art which may be used for the production of polyclonal antibodies to polypeptides of interest as disclosed herein. For the production of polyclonal antibodies, various host animals, including but not limited to rabbits, mice, rats, sheep, goats, etc, can be immunized by injection with a polypeptide, or fragment or analogue thereof. Further, the polypeptide or fragment or analogue thereof can be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Screening for the desired antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary antibody. Alternatively, the primary antibody may be detected by virtue of its binding with a secondary antibody or reagent which is appropriately labelled. A variety of methods are known in the art for detecting binding in an immunoassay and are within the scope of the present invention.

The antibody or fragment thereof raised has binding affinity for the polypeptide. Preferably, the antibody or fragment thereof has binding affinity or avidity greater than about 10⁵ M⁻¹, more preferably greater than about 10⁶ M⁻¹, more preferably still greater than about 10⁷ M⁻¹ and most preferably greater than about 10⁸ M⁻¹.

In terms of obtaining a suitable amount of an antibody according to the present invention, one may manufacture the antibody(s) using batch fermentation with serum free medium. After fermentation the antibody may be purified via a multistep procedure incorporating chromatography and viral inactivation/removal steps. For instance, the antibody may be first separated by Protein A affinity chromatography and then treated with solvent/detergent to inactivate any lipid enveloped viruses. Further purification, typically by anion and cation exchange chromatography may be used to remove residual proteins, solvents/detergents and nucleic acids. The purified antibody may be further purified and formulated into 0.9% saline using gel filtration columns. The formulated bulk preparation may then be sterilised and viral filtered and dispensed.

In a related aspect, the invention may feature a monoclonal antibody, or an Fab, (Fab)₂, scFv (single chain Fv), dAb (single domain antibody), bi-specific antibodies, diabodies and triabodies, or other immunologically active fragment thereof (eg., a complementarity-determining region). Such fragments are useful as immunosuppressive agents. Alternatively, the antibody of the invention may have attached to it an effector or reporter molecule. For instance, an antibody or fragment thereof of the invention may have a macrocycle, for chelating a heavy metal atom, or a toxin, such as ricin, attached to it by a covalent bridging structure. In addition, the Fc fragment or CH₃ domain of a complete antibody molecule may be replaced or conjugated by an enzyme or toxin molecule, such as chelates, toxins, drugs or prodrugs, and a part of the immunoglobulin chain may be bonded with a polypeptide effector or reporter molecule, such as biotin, fluorochromes, phosphatases and peroxidases. Bispecific antibodies may also be produced in accordance with standard procedures well known to those skilled in the art.

The present invention further contemplates genetically modifying the antibody variable and/or constant regions to include effectively homologous variable and constant region amino acid sequences. Generally, changes in the variable region will be made to improve or otherwise modify antigen binding properties of the antibody or fragment thereof. Changes in the constant region will, in general, be made in order to improve or otherwise modify biological properties, such as complement fixation, interaction with membranes, and other effector functions.

In the present context, effectively homologous refers to the concept that differences in the primary structure of the variable region of the antibody or fragment thereof may not alter the binding characteristics of the antibody or fragment thereof. Changes of amino acids are permissible in effectively homologous sequences so long as the resultant antibody or fragment thereof retains its desired property.

Amino acid changes in the polypeptide or the antibody or fragment thereof may be effected by techniques well known to persons skilled in the relevant art. For example, amino acid changes may be effected by nucleotide replacement techniques which include the addition, deletion or substitution of nucleotides, under the proviso that the proper reading frame is maintained. Exemplary techniques include random mutagenesis, site-directed mutagenesis, oligonucleotide-mediated or polynucleotide-mediated mutagenesis, deletion of selected region(s) through the use of existing or engineered restriction enzyme sites, and the polymerase chain reaction.

Also included within the scope of the present invention are alternative forms of inhibition of expression of polypeptides and polynucleotides disclosed herein, including, for example, small molecule or other non-nucleic acid or non-proteinaceous inhibitors. Such inhibitors may be identified by those skilled in the art by screening using routine techniques.

Compositions and Methods of Treatment

Polypeptides, polynucleotides and inhibitor compounds of the present invention may be administered as compositions either therapeutically or preventively. In a therapeutic application, compositions are administered to a patient already suffering from a disease, in an amount sufficient to cure or at least partially arrest the disease and its complications. The composition should provide a quantity of the compound or agent sufficient to effectively treat the patient.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.1 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

These compositions can be administered by standard routes. In general, the compositions may be administered by the parenteral (e.g., intravenous, intraspinal, subcutaneous or intramuscular), oral or topical route. Typically, administration is by the parenteral route.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The topical formulations of the present invention, comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which are incorporated herein by reference.

Those skilled in the art will appreciate that the compositions may be administered as part of a combination therapy approach to the treatment of MS, employing one or more of the compositions disclosed herein in conjunction with other therapeutic approaches to MS treatment. For such combination therapies, each component of the combination may be administered at the same time, or sequentially in any order, or at different times, so as to provide the desired therapeutic effect. When administered separately, it may be preferred for the components to be administered by the same route of administration, although it is not necessary for this to be so. Alternatively, the components may be formulated together in a single dosage unit as a combination product. Suitable agents which may be used in combination with the compositions of the present invention will be known to those of ordinary skill in the art. For example, the current main therapies for MS include interferon-β and glatiramer acetate (formerly called Copolymer-1 or COP-1), with many other therapies used to relieve the various symptoms of MS. In addition, monoclonal antibodies have been developed which target MS-associated antigens.

The present invention will now be described with reference to specific examples, which should not be construed as in any way limiting the scope of the invention.

### EXAMPLES

### General Methods

Blood Samples:

Whole blood was collected from 12 patients with chronic progressive disease, diagnosed according to McDonald's criteria (McDonald et al. (2001)); 6 primary progressive MS patients (4 females, 2 males) and 6 secondary progressive patients (3 females, 3 males). In addition, 20 female healthy controls (ages 20-50) gave blood for the reference sample, and 5 male healthy controls (ages 20-50) gave blood for the control sample. Blood was collected into PAX (Qiagen) vacutainer tubes and RNA extracted according to the manufacturer's instructions.

Microarrays:

RNA was amplified for one round using the Riboamp amplification kit (Geneworks). One microgram of MS amplified mRNA (aRNA) and one microgram of control pool aRNA was prepared and labeled using both the CyScribe Post-labeling Kit (Amersham) and Qiaquick columns (Qiagen). The labeled aRNA was hybridized to an 8K cDNA human microarray (Australian Genome Research Centre, Victoria, Australia) and the arrays scanned (GenePix 4000B scanner, Axon Instruments). The arrays were analysed using the software program R (www.bioconductor.org), and Acuity V4.0. Two arrays, including a dye swap array were hybridized per patient, as well as for the individual controls versus the reference pool, to provide a three-way design (Yang and Speed (2002)). The data was normalized (global loess) and all spots with an intensity less than 100 were filtered out of the data sets as such spots were considered unreliable. SAM (Significance Analysis of Microarrays) was used to compare relative detection of the mRNA from each gene within each sample (Tusher et al. (2001)). Pathway representation in the dysregulated group of genes was examined using GOstat with the Benjamini correction for multiple comparisons (Beissbarth and Speed (2004)).

Genotyping:

CD127 genotyping was performed as previously described (Teutsch et al. (2003)). Transmission distortion in 216 families (120 RRMS, 78 SPMS, 18 PPMS) was assessed (TDT test, EASYTDT website); and the association of SNPs and haplotypes with MS was evaluated in 182 ethnically matched controls and 363 MS cases (192 RRMS, 108 SPMS, 63 PPMS) using two tailed Fisher's Exact tests. Details of the MS DNA bank have been previously reported (Ban et al. (2003)).

RT-PCR:

cDNA was prepared from patient and reference pool total mRNA using standard methods. CD127 mRNA levels were assayed using Sybr green and primers spanning intron 7 of CD127. Primer sequences were 5′-CATCTTTGTAAGAAACCAAG-3′ (SEQ ID NO:9), 5′-TGGCAGTCCAGGAAACTTTC-3′ (SEQ ID NO:10). cDNA levels were measured using picogreen (Whelan et al. (2003)). ΔCT was used to measure comparative amplification (Livak and Schmittgen (2001)), and normalised against starting material.

CD127 Gene Expression:

The 6 PPMS patients, 6 SPMS patients and 17 controls, as well as an additional 8 control individuals, were genotyped for CD127 promoter alleles as previously described (Teutsch et al. (2003)). The PCR primers used to amplify cDNA samples were specific for CD127 mRNA membrane-bound and soluble splice variants, for the CD127 exon 8 amino acid residue 336 (aa336) alleles (Korte et al. (2000)) and for CD127 exon 2 amino acid residue 46 (aa46) alleles. The PCR primer sequences were CD127X2F: 5′-TGGAGAAAGTGGCTATGCTCA-3′ (SEQ ID NO:11) and CD127X2R: 5′-CAACCTTCACACATATATTGCTC-3′ (SEQ ID NO:12). The aa336 and aa46 alleles were in complete linkage disequilibrium with the promoter alleles at nucleotides −504 and −449, respectively. cDNA primer extension assays using the SNaPshot system (Applied Biosystems, Foster City, Calif., USA) were designed, involving three SNaPshot extension primers. These primers were designed to distinguish the CD127 exon 8 aa336 (A/G) SNP allele, with sequence 5′-AGCTCCAACTGCCCATCTGAGGATGTAGTC-3′ (SEQ ID NO: 13), and the exon 2 aa46 (C/T) SNP allele, with sequence 5′-GTGCTTTTGAGGACCCAGATGTCAACA-3′ (SEQ ID NO:14), in heterozygous individuals, and the CD127 soluble isoform with sequence 5′-TCCAGAGATCAATAATAGCTCAGG-3′ (SEQ ID NO:15) in individuals with representative CD127 genotypes. All reactions were performed in triplicate and means and standard errors were obtained for each individual. For the CD127 aa46 and aa336 SNaPshot reactions, the ratio of fluorescence peak heights of each allele in heterozygotes was calculated. SNaPshot reactions for aa46 and aa336 alleles were also performed in triplicate on representative control genomic DNA samples to correct for any biases in allelic amplification. The mean of the ratio of SNaPshot peaks was used as a correction factor by which all aa46 and aa336 SNaPshot cDNA ratios were divided. Mean cDNA ratios of expression were compared between MS patients and controls using the unpaired t-test (Graph Pad Quick Calcs—http://graphpad.com/quickcalcs) to obtain p-values. Mean cDNA ratios of expression were compared with genomic DNA ratios using the Mann-Whitney U-test (SPSS Inc., Chicago, Ill., USA). The fluorescence peak height ratios of CD127 mRNA splice variants were calculated and mean cDNA ratios of expression were correlated with CD127 promoter genotypes using the unpaired t-test.

### Example 1

### Shared Expression Profiles in PPMS and SPMS

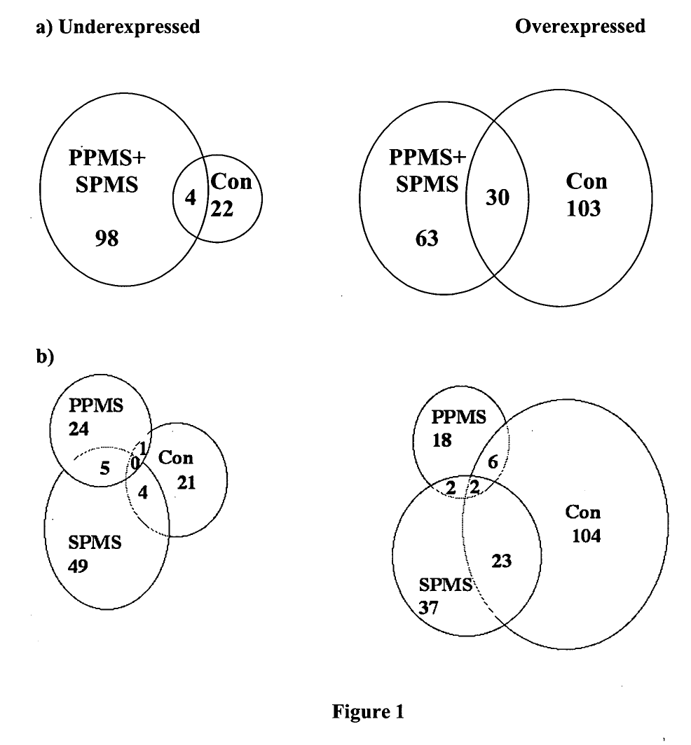

When both PPMS and SPMS groups were combined (PPMS+SPMS) and compared to the reference sample, with SAM set to a false discovery rate of just <1, 102 genes were found to be under-expressed and 93 genes over-expressed in the combined PPMS+SPMS group (FIG. 1a). Four of the 102 were also under-expressed in the control group compared to the reference group, and 30 of the 93 over-expressed genes were also shared. These 30 were removed from the list, leaving 98 and 63 genes dysregulated (FIG. 1a).

Biochemical pathways over-represented in the PPMS+SPMS sets were identified using GOstat (www.wehi.edu.au) with the Benjamini correction for multiple testing, and are listed in Table 1. Highlighted genomic locations are those within 1 MB of markers associated with MS in the GAMES study (Ban et al. (2003)).

Two pathways were significantly over-represented in the over-expressed group: amino acid phosphorylation, and response to stimuli. Amino acid phosphorylation activates many cellular responses, notably cell adhesion and migration. Genes from this group were mainly over-expressed in SPMS patients, and this pathway was the most over-expressed during comparison between the SPMS and reference groups. Genes from the response to stimuli pathway were up-regulated in both groups. Arachidonate 5-lipoxygenase (ALOX5) enables the first step in leukotriene synthesis, and has been previously shown to be up-regulated in macrophages in MS and in EAE, the mouse model of MS, in microarray studies (Whitney et al. (2001)). Leukotrienes have numerous pro-inflammatory functions, including increasing vascular permeability. Only the trinucleotide synthase pathway was significantly over-represented in the under-expressed genes, and this pathway was also down-regulated in the comparison between PPMS and reference groups. ATP synthesis in the mitochondria is fundamental to cellular activation and proliferation, processes which seem to be down-regulated in PPMS (see Example 2 and Table 3 below).

TABLE 1
Biochemical pathways over-represented in dysregulated
PPMS + SPMS genes
PPMS + SPMS Over-expressed

| Reponse to stimuli | | Amino acid phosphorylation | |
|---|---|---|---|
| P = 0.013 | | P= 0.014 | |
| CD53 | 1p13 | MAP4K4 | 2q11 |
| GBP2 | 1p22 | STAT1 | 2q32 |
| IL18RAP | 2p24 | GSK3B | 3q13.3 |
| IL1R2 | 2q12 | FGFR4 | 5q35 |
| STAT1 | 2q32 | SRPK1 | 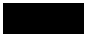 |
| HLA-G | 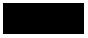 | SLK | 10q24.3 |
| HLA-DOA | 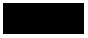 | PAK1 | 11q13 |
| PPPIR10 | 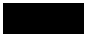 | PRKAR1A | 17q23 |
| DEFA4 | 8p23 | ROCK1 | 18q11 |
| ALOX5 | 10q11.2 | MAPK1 | 22q11 |
| CD97 | 19p13 | RPSP6KA3 | Xp22 |
| BP1 | 20q11 | | |
| MX2 | 21q22.3 | | |
| XBP1 | 22q12 | | |

PPMS + SPMS Under-expressed

| Triphosphate synthesis | |
|---|---|
| P = 0.004 | |
| NME6 | 3p21 |
| ATP51 | 4p16.3 |
| NME2 | 17q21.3 |
| ATP50 | 21q22.1 |
| RP2 | Xp11 |

When PPMS and SPMS were compared separately to the reference group, there was only a small overlap in shared dysregulated genes (FIG. 1b, Table 2).

TABLE 2
Dysregulated genes in SPMS and PPMS
compared to the Reference sample

| | | Chromosomal |
|---|---|---|
| Accession No | Name | Location |
| Overexpressed | | |
| R42600 | matrix metalloproteinase 17 | 12q24.3 |
| | (membrane-inserted) | |
| AA282134 | glutaminyl cyclase | 2p22.3 |
| Underexpressed | | |
| AA663981 | immunoglobulin heavy locus | 14q32.33 |
| W68403 | integrin, beta 2 (CD18) | 21q22.3 |
| AA486418 | transcription factor RAM2 | 7p15.3 |
| H37827 | pipecolic acid oxidase | 17q11.2 |
| N53169 | apolipoprotein C-III | 11q23.1 |
| AA454610 | Mixed-lineage leukaemia | 17q21 |

Metalloproteases have been implicated in MS through their importance in T cell infiltration of the brain. MMP17 is a membrane bound metalloprotease known to be able to degrade components of the extracellular matrix and activate TNFα, a type 1 cytokine (English et al. (2000)). Low levels of the 5 genes under-expressed in both PPMS and SPMS would not obviously be expected to contribute to MS pathogenesis. However, their expression levels may reflect altered balances with other gene products which contribute to disease. For example, CD18 enables myeloid cell adhesion through heterodimerisation with the CD11 proteins. Although adhesion is important in migration of leukocytes across the blood-brain prior, it is also required for binding of autoreactive T cells by regulatory T cells (Grossman et al. 2004)). The latter process may be more significant in PPMS/SPMS pathogenesis.

### Example 2

### Different Expression Profiles in MS Subtypes

If SPMS and PPMS are compared to each other, 25 genes are under-expressed in PPMS, and none is over-expressed (Table 3). Most of the genes under-expressed in PPMS compared to SPMS were also under-expressed in PPMS compared to the healthy controls, but the differences were greater between PPMS and SPMS. These data suggest that although there were shared differences between SPMS and PPMS compared to the control groups, the most dysregulated genes in each were different for PPMS and SPMS.

A striking result is the number of ribosomal genes under-expressed in PPMS (9 out of 25, P<10⁻⁴). These genes are usually regulated in concert (Grewal et al. (2004)), and they might be expected to cluster in gene expression profiles. Many transcription factors were also under-expressed (6 out of 25), and some of these are known to affect ribosomal gene regulation (MAX, PUR). The latter binds to purines, and a functionally related transcription factor, PU.1, has recently been shown to regulate CD127 expression (Xue et al. (2004)). These data point to a generalised down-regulation of genes important in cell proliferation and activation in PPMS.

TABLE 3
Genes dysregulated between PPMS and SPMS

| Genbank | | Chromosomal |
|---|---|---|
| Accession No. | Name¹ | Location |
| AA485865 | CD127 (Interleukin-7 receptor α chain) | 5p13 |
| AA633768 | 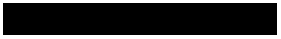 | 3q12 |
| AI005610 | 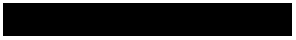 | 19q13.3 |
| AA488900 | Rap guanine nucleotide exchange factor | 4q32.1 |
| AA463631 | signal recognition particle 72kDa | 4q11 |
| T63324 | major histocompatibility complex, class II, DQ alpha 2 | 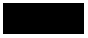 |
| AI936175 | 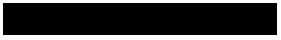 | 8q12 |
| AA634008 | 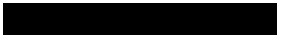 | 5q14.1 |
| H56944 | splicing factor, arginine/serine-rich 11 | 1p31 |
| R43544 | 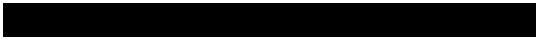 | 7p13 |
| AA447515 | MAX dimerization protein 4 | 4p16.3 |
| W35411 | neuro-oncological ventral antigen 2 | 19q13.3 |
| AA868008 | histone 1, H4f | 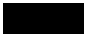 |
| AA629641 | 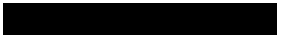 | 11p15 |
| AA446108 | endoglin (Osler-Rendu-Weber syndrome 1) | 9q33-q34.1 |
| H23422 | 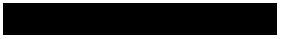 | 9q34 |
| AA862813 | cytochrome c oxidase subunit 8A | 11q12-q13 |
| AA132226 | chromobox homolog 3 | 7p15.2 |
| H72918 | bromodomain containing 2 | 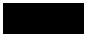 |
| N64862 | FYN binding protein (FYB-120/130) | 5p13.1 |
| AI928745 | POU domain, class 3, transcription factor 4 | Xq21.1 |
| AA912448 | ELK3, ETS-domain protein (SRF accessory protein 2) | 12q23 |
| AA668301 | 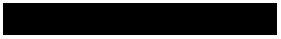 | 19q13.1 |
| H46425 | purine-rich element binding protein A | 5q31 |
| AA629897 | 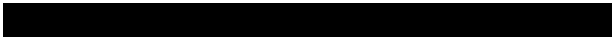 | 3p21.3 |

¹Gene names highlighted in grey are those encoding ribosomal proteins, and underlined are transcription factors. Highlighted genomic locations are those within 1MB of markers associated with MS in the GAMES study.

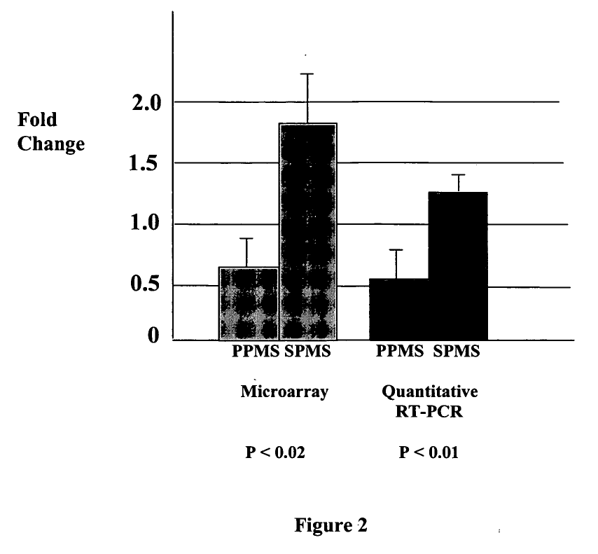

CD127 was down-regulated in PPMS and up-regulated in SPMS. It has been previously identified as up-regulated in RRMS (Ramanathan et al. (2001)) and was also detected as differentially regulated between PPMS and SPMS using RT-PCR (FIG. 2). The genomic region encoding CD127 has been previously associated with MS, and IL-7 and its receptor are vital for T cell maturation and proliferation. Competition for scarce IL-7 between cell types may result in reduced survival of protective cells in PPMS, such as regulatory T cells. Further, as CD127 is also a component of the receptor for thymic stromal lymphopoietin (TSLP), a cytokine which activates CD11c+ dendritic cells, and results in their Th2 cytokine production (Soumelius et al. (2002)), reduction in levels of the TSLP receptor may cause a Th1 skew in PPMS.

### Example 3

### Population Association of Allelic Polymorphisms in Promoter Regions of Differentially Expressed Genes

Genes which are differentially expressed may 1) contribute directly to MS development and progression, or 2) be an effect of MS pathogenesis, for example, as part of the homeostatic process, or 3) be unrelated to MS, for example, detected by chance or be dysregulated through epigenetic effects of genes which are dysregulated due to 1) or 2). A telling way to distinguish between these possibilities is to identify those genes which are differently expressed due to genetic variation in their promoters—if such promoter SNPs are associated with MS (detected by genotyping), and their gene product is also associated (detected by microarray analysis), then the gene is more likely to be contributory to MS development. The inventors sought genes encoded within 1 mb of markers most associated with MS in the GAMES study (Ban et al. (2003)) in the set of dysregulated genes. Only the genes of the MHC cluster on 6p21.3 were encoded in these regions. Numerous genes from 6p21 were detected in over and under-expressed groups (see Tables 1-3 above).

The inventors have examined the putative promoter region of CD127 by pooled and individual DNA sequencing, and identified several common polymorphisms (Table 4). The polymorphisms were not aberrantly represented in the CPMS patients used in the microarray experiments.

TABLE 4
Common promoter polymorphisms and haplotypes in putative
promoter regions of CD127

| CD127 | | | | | |
|---|---|---|---|---|---|
| Haplotype | −1085 | −504 | −449 | aa46 | aa336 |
| 1 | G | C | A | C | G |
| 2 | G | T | G | T | A |
| 3 | G | T | A | C | A |
| 4 | T | T | A | C | A |

### Example 4

### CD127 Population Association Study

Previous studies have failed to detect an association between the CD127 polymorphisms and MS (Teutsch et al. (2003)). As the inventors have now identified opposite expression levels in PPMS and SPMS, they tested for the association of clinical phenotype with the CD127 polymorphisms (Tables 5 and 6).

TABLE 5
Transmission of CD127 alleles in trio families

| IL7R-504 | PPMS | SPMS | PPMS/SPMS | RRMS |
|---|---|---|---|---|
| T/C transmitted | 10/3 | 21/28 | 31/31 | 49/55 |
| No. Families | 18 | 78 | 96 | 120 |
| TDT (P) | 0.05 | 0.32 | 1.0 | 0.56 |

TABLE 6
Frequency of CD127 - 504 T/C (C is tag for GCA
promoter haplotype) alleles and genotypes in MS cases
(according to clinical phenotype) and controls

| | | SPMS | RRMS | Controls* |
|---|---|---|---|---|
| | PPMS (N = 63) | (N = 108) | (N = 192) | (N = 182) |
| SNP | | | | |
| C | 25 | 57 | 108 | 100 |
| T | 101 | 159 | 276 | 264 |
| % MAF¹ | 19** | 26 | 28 | 27 |
| Genotype | | | | |
| CC² | 5 (8)*** | 10 (9) | 13 (7) | 8 (4) |
| CT² | 15 (24)*** | 37 (34) | 82 (43) | 84 (46) |
| TT² | 43 (69) | 61 (56) | 97 (50) | 90 (50) |

¹MAF = minor allele frequency
²Percentages in parentheses
*from Teutsch et al (2003)
**P = 0.07, alleles (Fisher's Exact, 2 tailed test, PPMS cf Controls)
***P = 0.01, carriers (Fisher's Exact, 2 tailed test, PPMS cf Controls)

In trios, the −504T allele was significantly over-transmitted in PPMS, and the C allele was more common in SPMS, though not significantly. In the case control study (Table 6), there was over-representation of the −504T allele in PPMS (P=0.01), and a non-significant trend towards over-representation of the C allele in SPMS. The C allele is a marker for the GCA haplotype, which is thus under-represented in PPMS. The opposite associations in PPMS and SPMS mask the associations of CD127 with MS when these two are combined, highlighting the confounding effect of heterogeneity in other previous association studies.

### Example 5

### CD127 Expression from Different Haplotypes

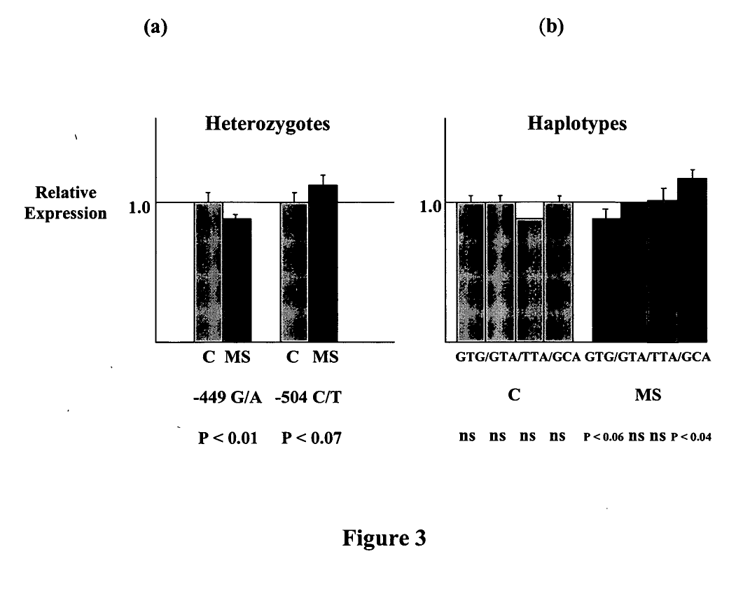

The inventors investigated the in vivo expression of CD127 using cDNA primer extension assays. Haplotype tag polymorphisms are present in the coding region of CD127: the SNP at aa46 tags promoter haplotype GTG (if ‘T’) and GCA, TTA and GTA if ‘C’ (Table 4). The SNP at aa336 tags GCA if ‘G’ and the other 3 haplotypes if ‘A’. By comparing the proportion of each SNP in the mRNAs collected ex vivo, the product from each promoter haplotype can be compared. In healthy controls, no difference in promoter haplotype expression was detected, but in PPMS/SPMS patients a small but significant difference was detected in haplotype expression (FIGS. 3a and 3b), with promoter haplotype GCA being the high expressor.

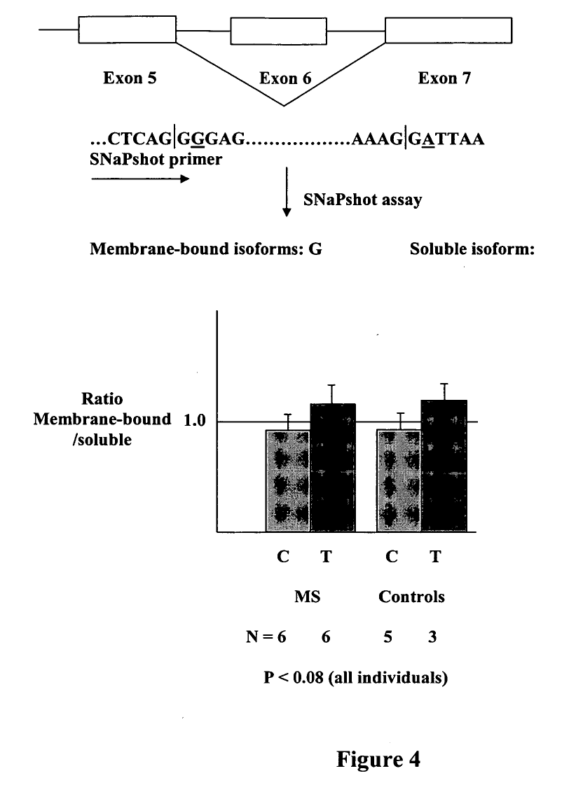

An isoform of CD127, in which exon 6 is spliced out, makes up about 10% of the message in healthy controls. Relative expression of this isoform from the different haplotypes can be measured using an oligo at the exon 6 splice site, and comparing the expression levels of the ‘G’ corresponding to full length cDNA, or ‘A’ corresponding to the soluble isoform mRNA, as in the cDNA primer extension assay, for known genotypes. There was no significant difference in proportion of soluble CD127 between the GCA and other haplotypes, in healthy controls or MS (FIG. 4), although there was a trend (p=0.085) between the −504C (GCA haplotype) and more soluble CD127 in both. Local variation and variation in cell subsets in production of soluble CD127 could lead to differential cell activation, maturation, and proliferation. Once again, a more targeted approach to measurement of haplotype effect on mRNA expression, through selection of different cell subsets in health and disease, would be required to establish its importance in MS, an effort that would certainly be warranted if the haplotypic association reported here was confirmed in independent studies.

### Example 6

### The Effect of CD127 Genotype on CD127 Expression

Having demonstrated that CD127 mRNA expression is lower in PPMS, and that the CD127 genotypes more common in PPMS are low expressors of CD127 mRNA, the effect of CD127 genotype on CD127 expression was then investigated.

Approximately 45 mls of blood was taken from 10 PPMS patients and 18 ethnically and sex matched controls. Buffy coat and plasma were then taken for separate freezing, regulatory T cell purification and antibody staining. 100 μl of buffy coat was stained with 50 μl blocking antibody (12CA5) and 20 μl of anti-CD25, CD14 and CD56 (FITC). Cells were stained with 20 μl of anti-CD127 (PE), anti-CD3 and -CD4 (PerCP), and a control tube was set up with anti-IgG1 (FITC, PE and PerCP), and then analysed by flow cytometry for cell type, cell number and CD127 expression. Cells were incubated for 30 minutes, and washed twice before fixing and running on a FACScan (BD Biosciences) 3 colour Flow Cytometer in duplicate.

For analysis of data acquired by flow cytometry, cell population isolation was undertaken via regulatory T cell, NK and NKT cell and monocyte protocols using Cell Quest software. Cells were first isolated through forward verses side scatter profiles, then gated based on side scatter and known antibody expression (eg. CD25 for T cells, CD14 for monocytes and CD56 for NK/NKTs). Dead cells were also gated and removed using Boolean tools within the software, and cell numbers and CD127 expression determined for each cell type.

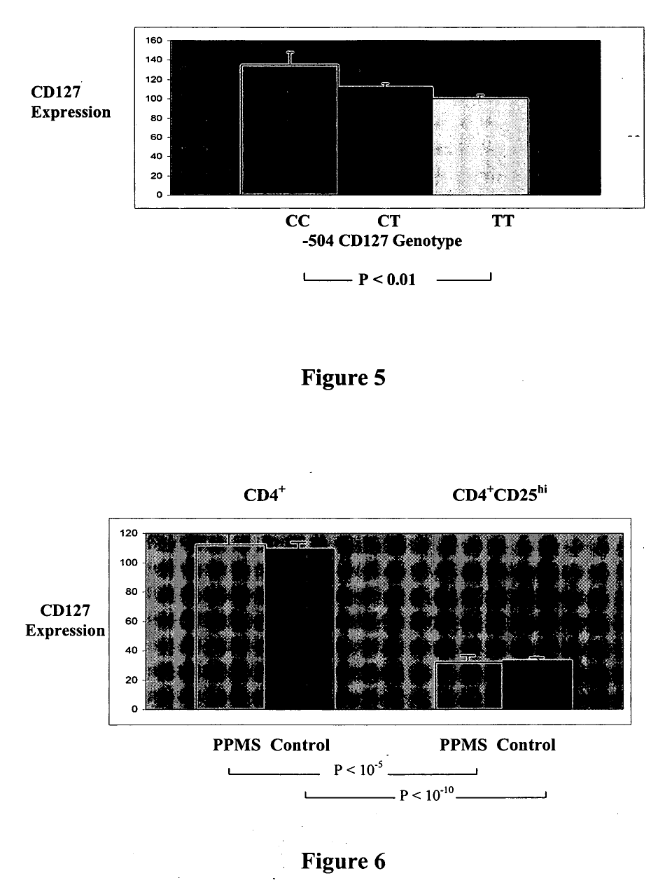

FACS analysis demonstrated that the CD127 −504 CT and TT genotypes prevalent in PPMS have lower CD127 protein expression in CD4+ T cells (FIG. 5). These data are in accord with the concept that a consequence of these genotypes in PPMS is reduced CD127 expression.

### Example 7

### CD127 Expression is Reduced in Treg and NKT Cells

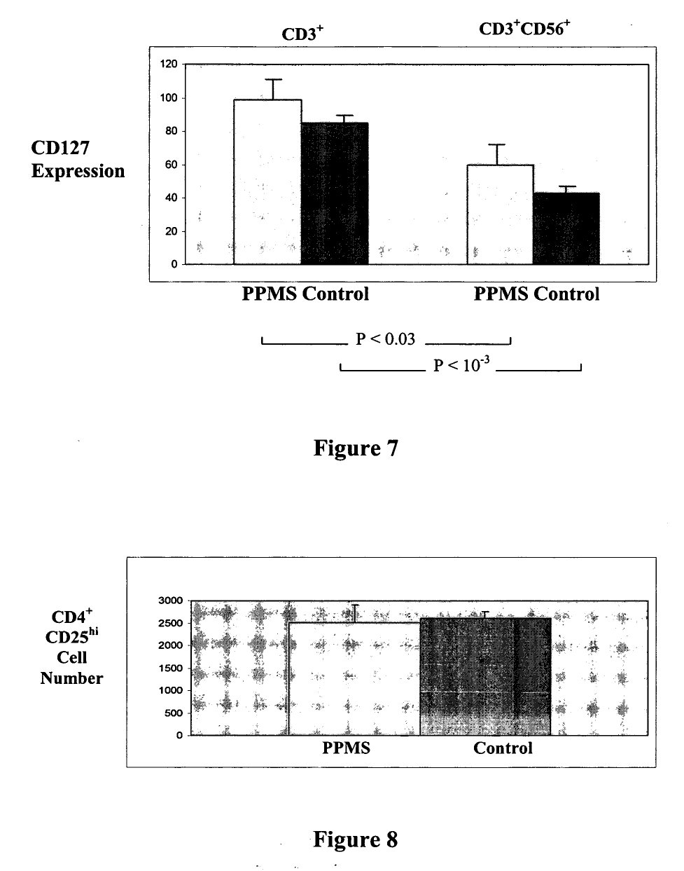

Further FACS analysis of the samples described above in Example 6 demonstrated that Tregs (CD4⁺ CD25^{bi}) had less CD127 expression than other T cells (CD4⁺) (FIG. 6), as did NKT cells (CD3⁺ CD56⁺) compared to other T cells (CD3⁺) (FIG. 7). This result is consistent with the hypothesis that Treg and NKT cells have reduced CD127 expression and so are less able to compete with other T cells for limited IL7. In individuals with the lower expressing CD127 genotype, the reduced competitiveness of the Tregs and NKTs would be exacerbated.

### Example 8

### Analysis of Treg and NKT Cell Number in PPMS

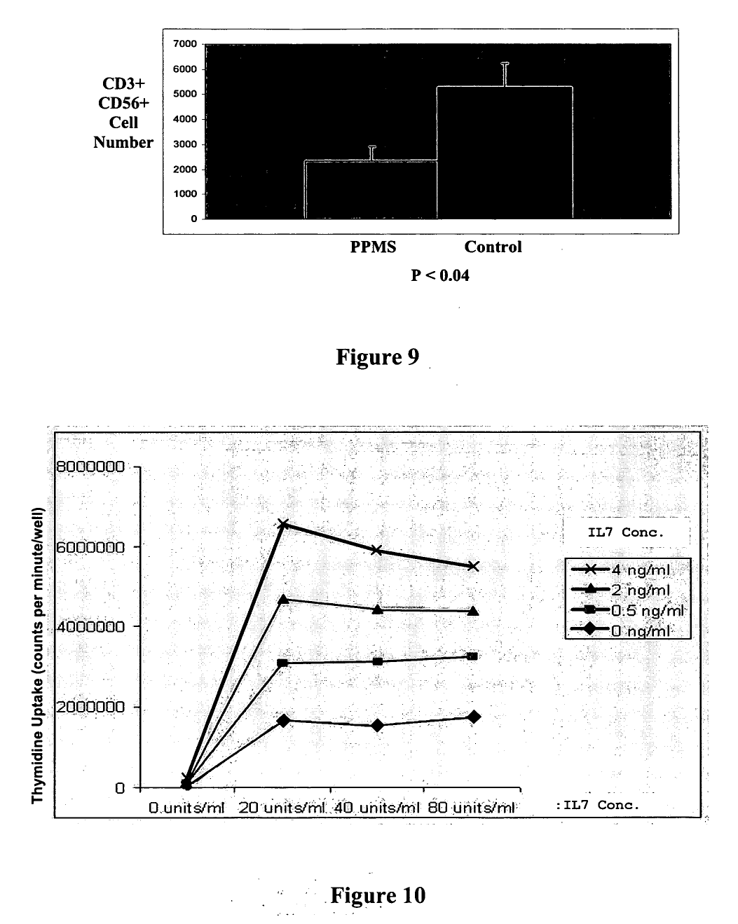

Using the same strategy and FACS analysis as described above in Example 6, it was found that numbers of CD4⁺ CD25^{hi} (Tregs) were not different between PPMS and control samples (FIG. 8). However, numbers of CD3⁺ CD56⁺ (NKTs, also with regulatory function) were different between PPMS and control samples (FIG. 9). The lack of difference in Treg cell numbers between PPMS and control samples may be interpreted on the basis that impairment of Treg function, as opposed to Treg number, may nevertheless be important in the pathogenesis of PPMS.

### Example 9

### Effect of IL7 on Proliferation of T cell Subsets

In order to investigate the effect of IL7 on the proliferation of various T cell subsets in both healthy controls and PPMS patients, a series of cell proliferation studies was undertaken.

In the first of these studies, blood was removed from a healthy control subject. CD4⁺ CD25⁺ cells were purified from Ficoll isolated PBMCs using MACS Separators (Miltenyi Biotec). The cell purity of the Treg fraction (CD4⁺ CD25⁺) was determined to be 95.3%. The proportion of CD4⁺ CD25⁻ cells was found to be 1.9%. Purified cells were cultured in X-Vivo 15 (Cambrex Bioscience) in round-bottom 96 well plates containing 7.5 μl Dynal anti-CD3/anti-CD28 beads diluted in 3 mls of medium, and dispensed at 50 μl per well, with 10⁴ Treg cells per well and amounts of IL-2 and/or IL-7 as outlined in Table 7 below.

TABLE 7
Amounts of IL-2 and/or IL-7 used in cell proliferation studies

| | 0 U IL-2/ml | 20 U IL-2/ml | 40 U IL-2/ml | 80 U IL-2/ml |
|---|---|---|---|---|
| 0 μg/ml IL-7 | Beads + cells | Beads + cells | Beads + cells | Beads + cells |
| 0.5 μg/ml IL-7 | Beads + cells | Beads + cells | Beads + cells | Beads + cells |
| 1 μg/ml IL-7 | Beads + cells | Beads + cells | Beads + cells | Beads + cells |
| 2 μg/ml IL-7 | Beads + cells | Beads + cells | Beads + cells | Beads + cells |

On days 2, 4 and 6 of culture, 100 μl of medium was removed from each well and wells were then replenished with respective concentrations of IL-2 and IL-7. Cells were then pulsed with ³H-thymidine (0.5 μCi per well) on day 7. Cells were harvested and counted on day 8. The results are shown in FIG. 10, from which it can be seen that the proliferation of cultured cells in the presence of IL-2 was augmented with exposure to increasing concentrations of IL7, thereby indicating that IL7 can work synergistically with IL2 to increase proliferation of Treg.

In the second study, blood was removed from a PPMS patient and CD4⁺ CD25⁺ cells (Tregs) purified from Ficoll isolated PBMCs using MACS Separators (Miltenyi Biotec). The purity of the CD4⁺ CD25⁺ (Treg) fraction was determined to be 81.5%, with the CD4⁺ CD25⁻ fraction determined to be 17.7%. Cells were cultured in X-Vivo 15 (Cambrex Bioscience) and grown in round-bottom 96 well plates containing 7.5 μl of Dynal anti-CD3/anti-CD28 beads in 3 mls of medium, dispensed at 50 μl/well, and 10⁴ Treg cells per well. Duplicate wells were plated containing:
1. Medium only;
2. IL-2 only [20 units per ml (final concentration)];
3. IL-7 only [1 ng per ml (final concentration)]; or
4. IL-2 [20 units per ml (final concentration)] and IL-7 [1 ng per ml (final concentration)].

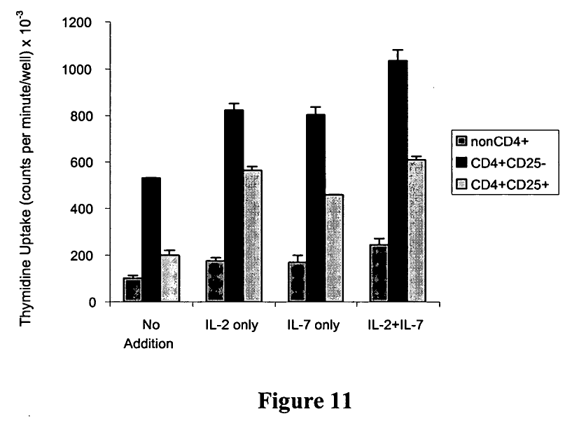

On day 2, 100 μl of medium was removed and replaced with fresh cytokines. Cultures were pulsed with ³H-thymidine at 0.5 μCi per well on day 3. Cells were harvested and counted on day 4. The results are shown in FIG. 11, demonstrating that IL7 causes proliferation of Tregs in vitro.

### Example 10

### Compositions for Treatment

In accordance with the best mode of performing the invention provided herein, specific preferred compositions are outlined below. The following are to be construed as merely illustrative examples of compositions and not as a limitation of the scope of the present invention in any way.

### Example 10(A)

### Composition for Parenteral Administration

A composition for parenteral injection could be prepared to contain 0.05 mg to 5 g of a suitable agent or compound as disclosed herein in 10 mls to 2 litres of 1% carboxymethylcellulose.

Similarly, a composition for intravenous infusion may comprise 250 ml of sterile Ringer's solution, and 0.05 mg to 5 g of a suitable agent or compound as disclosed herein.

### Example 10(B)

### Composition for Oral Administration

A composition of a suitable agent or compound in the form of a capsule may be prepared by filling a standard two-piece hard gelatin capsule with 500 mg of the agent or compound, in powdered form, 100 mg of lactose, 35 mg of talc and 10 mg of magnesium stearate.

### REFERENCES

Ban M, Sawcer S J, Heard R N, Bennetts B H, Adams S, Booth D, Perich V, Setakis E, Compston A, Stewart G J. (2003) A genome-wide screen for linkage disequilibrium in Australian HLA-DRB1*1501 positive multiple sclerosis patients. J Neuroimmunol. October;143(1-2):60-64.
Beissbarth T, Speed T. (2004) GOstat: Find statistically overrepresented Gene Ontologies within a group of genes. Bioinformatics. February 12 [Epub ahead of print]
Chabas D, Baranzini S E, Mitchell D, Bernard C C, Rittling S R, Denhardt D T, Sobel R A, Lock C, Karpuj M, Pedotti R, Heller R, Oksenberg J R, Steinman L. (2001) The influence of the proinflammatory cytokine, osteopontin, on autoimmune demyelinating disease. Science. November 23;294(5547):1731-1735.
Compston A, Sawcer S. (2002) Genetic analysis of multiple sclerosis. Curr Neurol Neurosci Rep. May;2(3):259-266.
Dyment D A, Ebers G C, Sadovnick A D. (2004) Genetics of multiple sclerosis. Lancet Neurol. February;3(2):104-110.
English W R, Puente X S, Freije J M, Knauper V, Amour A, Merryweather A, Lopez-Otin C, Murphy G. (2000) Membrane type 4 matrix metalloproteinase (MMP17) has tumor necrosis factor-alpha convertase activity but does not activate pro-MMP2. J Biol. Chem. May 12;275(19):14046-14055.
Grewal R, Stepczynski J, Kelln R, Erickson T, Darrow R, Barsalou L, Patterson M, Organisciak D T, Wong P. (2004) Coordinated changes in classes of ribosomal protein gene expression is associated with light-induced retinal degeneration. Invest Ophthalmol Vis Sci. November;45(11):3885-3895.
Grossman W J, Verbsky J W, Barchet W, Colonna M, Atkinson J P, Ley T J. (2004) Human T regulatory cells can use the perforin pathway to cause autologous target cell death. Immunity. October;21(4):589-601.
Korte A, Kochling J, Badiali L, Eckert C, Andreae J, Geilen W, Kebelmann-Betzing C, Taube T, Wu S, Henze G, Seeger K. (2000) Expression analysis and characterization of alternatively spliced transcripts of human IL-7Ralpha chain encoding two truncated receptor proteins in relapsed childhood all. Cytokine. November;12(11):1597-1608.
Livak K J, Schmittgen T D. (2001) Analysis of relative gene expression data using real-time quantitative PCR and the 2(-Delta Delta C(T)) Method. Methods. December;25(4):402-408.
Lock C B, Heller R A. (2003) Gene microarray analysis of multiple sclerosis lesions. Trends Mol Med. December;9(12):535-541.
Lock C, Hermans G, Pedotti R, Brendolan A, Schadt E, Garren H, Langer-Gould A, Strober S, Cannella B, Allard J, Klonowski P, Austin A, Lad N, Kaminski N, Galli S J, Oksenberg J R, Raine C S, Heller R, Steinman L. (2002) Gene-microarray analysis of multiple sclerosis lesions yields new targets validated in autoimmune encephalomyelitis. Nat Med. May;8(5):500-508.
McDonald W I, Compston A, Edan G, Goodkin D, Hartung H P, Lublin F D, McFarland H F, Paty D W, Polman C H, Reingold S C, Sandberg-Wollheim M, Sibley W, Thompson A, van den Noort S, Weinshenker B Y, Wolinsky J S. (2001) Recommended diagnostic criteria for multiple sclerosis: guidelines from the International Panel on the diagnosis of multiple sclerosis. Ann Neurol July;50(1):121-7.
Pugliatti M, Sotgiu S, Rosati G. The worldwide prevalence of multiple sclerosis. (2002) Clin Neurol Neurosurg 104:182-91.
Ramanathan M, Weinstock-Guttman B, Nguyen L T, Badgett D, Miller C, Patrick K, Brownscheidle C, Jacobs L. (2001) In vivo gene expression revealed by cDNA arrays: the pattern in relapsing-remitting multiple sclerosis patients compared with normal subjects. J Neuroimmunol. June 1; 116(2):213-9.
Robertson N P, O'Riordan J I, Chataway J, Kingsley D P, Miller D H, Clayton D, Compston D A. (1997) Offspring recurrence rates and clinical characteristics of conjugal multiple sclerosis. Lancet. May 31;349(9065):1587-1590.
Soumelis V, Reche P A, Kanzler H, Yuan W, Edward G, Homey B, Gilliet M, Ho S, Antonenko S, Lauerma A, Smith K, Gorman D, Zurawski S, Abrams J, Menon S, McClanahan T, de Waal-Malefyt Rd R, Bazan F, Kastelein R A, Liu Y J. (2002) Human epithelial cells trigger dendritic cell mediated allergic inflammation by producing TSLP. Nat Immunol. July;3(7):673-80.
Sturzebecher S, Wandinger K P, Rosenwald A, Sathyamoorthy M, Tzou A, Mattar P, Frank J A, Staudt L, Martin R, McFarland H F. (2003) Expression profiling identifies responder and non-responder phenotypes to interferon-beta in multiple sclerosis. Brain. June;126(Pt 6):1419-1429.
Teutsch S M, Booth D R, Bennetts B H, Heard R N, Stewart G J. (2003) Identification of 11 novel and common single nucleotide polymorphisms in the interleukin-7 receptor-alpha gene and their associations with multiple sclerosis. Eur J Hum Genet. July;11(7):509-515.
Tusher V G, Tibshirani R, Chu G. (2001) Significance analysis of microarrays applied to the ionizing radiation response. Proc Natl Acad Sci USA. April 24;98(9):5116-5121.
Wandinger K P, Lunemann J D, Wengert O, Bellmann-Strobl J, Aktas 0, Weber A, Grundstrom E, Ehrlich S, Wemecke K D, Volk H D, Zipp F. (2003) TNF-related apoptosis inducing ligand (TRAIL) as a potential response marker for interferon-beta treatment in multiple sclerosis. Lancet. June 14;361(9374):2036-2043.
Whelan J A, Russell N B, Whelan M A. (2003) A method for the absolute quantification of cDNA using real-time PCR. J Immunol Methods. July;278(1-2):261-9.
Whitney L W, Ludwin S K, McFarland H F, Biddison W E. (2001) Microarray analysis of gene expression in multiple sclerosis and EAE identifies 5-lipoxygenase as a component of inflammatory lesions. J Neuroimmunol. December 3;121(1-2):40-8.
Xue H H, Bollenbacher J, Rovella V, Tripuraneni R, Du Y B, Liu C Y, Williams A, McCoy J P, Leonard W J. (2004) GA binding protein regulates interleukin 7 receptor alpha-chain gene expression in T cells. Nat Immunol. October;5(10):1036-44
Yang Y H, Speed T. (2002) Design issues for cDNA microarray experiments. Nat Rev Genet. August;3(8):579-88.

1. A method for treating CD127-low multiple sclerosis in a patient, the method comprising administering to the patient an effective amount of IL-7 or leukocytes treated with IL-7.

2. The method according to claim 1 wherein the IL-7 comprises the amino acid sequence as set forth in SEQ ID NO:1.

3. The method according to claim 1 wherein the IL-7 is administered by adoptive transfer of autologous leukocytes stimulated by contact with IL-7 in vitro.

4. The method according to claim 3 wherein the leukocytes are T-cells.

5. The method according to claim 1 wherein the IL-7 is administered in the form of a nucleic acid molecule encoding IL-7.

6. The method according to claim 5 wherein the nucleic acid molecule comprises the nucleotide sequence as set forth in SEQ ID NO:2.

7. A method for treating CD127-low multiple sclerosis in a patient, the method comprising administering to the patient an effective amount of leukocytes that have been induced to increase their cell surface expression of at least one receptor, a subunit of which is CD127.

8. The method according to claim 7 wherein the leukocytes are T-cells.

9. The method according to claim 7 wherein the receptor is either the IL-7 receptor and/or the TSLP receptor.

10. The method according to claim 7 wherein the leukocytes are obtained from the patient and are transformed with at least one nucleic acid molecule encoding one or more subunits of the IL-7 receptor and/or the TSLP receptor.

11. The method according to claim 10 wherein the at least one nucleic acid molecule comprises the nucleotide sequence set forth in SEQ ID NO:3, SEQ ID NO:4 or SEQ ID NO:5.

12. A method for treating CD127-low multiple sclerosis in a patient, the method comprising administering to the patient an effective amount of a nucleic acid molecule encoding at least CD127.

13. The method according to claim 12 further comprising administering to the patient an effective amount of a nucleic acid molecule encoding CD132.

14. The method according to claim 13 wherein the nucleic acid molecule encoding CD127 comprises the nucleotide sequence set forth in SEQ ID NO:3 and the nucleic acid molecule encoding CD132 comprises the nucleotide sequence set forth in SEQ ID NO:4.

15. The method according to claim 12, further comprising administering to the patient an effective amount of a nucleic acid molecule encoding the TSLP-R chain.

16. The method according to claim 15 wherein the nucleic acid molecule comprises the nucleotide sequence set forth in SEQ ID NO:5.

17. A method for treating CD127-low multiple sclerosis in a patient, the method comprising administering to the patient an effective amount of TSLP or leukocytes treated with TSLP.

18. The method according to claim 17 wherein the TSLP comprises the amino acid sequence as set forth in SEQ ID NO:6.

19. The method according to claim 17 wherein the TSLP is administered by adoptive transfer of autologous leukocytes stimulated by contact with TSLP in vitro.

20. The method according to claim 19 wherein the leukocytes are T-cells.

21. The method according to claim 17 wherein the TSLP is administered in the form of a nucleic acid molecule encoding TSLP.

22. The method according to claim 21 wherein the nucleic acid molecule comprises the nucleotide sequence as set forth in SEQ ID NO:7.

23. A method for treating CD127-high multiple sclerosis in a patient, the method comprising administering to the patient an effective amount of a non-functional form or homologue of IL-7 or TSLP.

24. A method for treating CD127-high multiple sclerosis in a patient, the method comprising administering to the patient an effective amount of a soluble form of the IL-7 receptor.

25. The method according to claim 24 wherein the soluble IL-7 receptor is administered as one or more polypeptide subunits.

26. The method according to claim 25 wherein the polypeptide subunit is CD127.

27. The method according to claim 24 wherein the soluble IL-7 receptor is administered as one or more nucleic acid molecules encoding polypeptide subunits of the soluble IL-7 receptor.

28. A method for treating CD127-high multiple sclerosis in a patient, the method comprising administering to the patient an effective amount of at least one inhibitor of one or more of: IL-7; TSLP; CD127; CD132; the TSLP-R chain; the IL-7 receptor; or the TSLP receptor.
